(19) United States
(12) Patent Application Publication
(10) Pub. No.: US 2024/0189535 A1
(43) Pub. Date: Jun. 13, 2024

(54) BREATHING GAS HUMIDIFIER

(71) Applicant: Drägerwerk AG & Co. KGaA, Lübeck (DE)

(72) Inventors: Ludger TAPPEHORN, Lübeck (DE); Christoph BRANDT, Lübeck (DE); Lena LUERS, Lübeck (DE); Maximilian HÜNERT, Lübeck (DE)

(21) Appl. No.: 18/530,327

(22) Filed: Dec. 6, 2023

(30) Foreign Application Priority Data
Dec. 7, 2022 (DE) 10 2022 132 441.9

(51) Int. Cl. A61M 16/16

(52) U.S. Cl. CPC A61M 16/16

(57) ABSTRACT

A breathing gas humidifier includes a water chamber, which has a first area for receiving water and a second area for receiving breathing gas. An inlet supplies breathing gas to the breathing gas humidifier, an outlet discharges conditioned breathing gas from the breathing gas humidifier, and a bubbler, including a bubbler element, has outlet ports. The breathing gas humidifier is configured to provide, in a first configuration, a fluidic connection between the inlet and the first area via the bubbler element to pass the breathing gas through the water to obtain the conditioned breathing gas. The breathing gas humidifier is further configured, in a second configuration, to provide a fluidic connection between the inlet and the second area to direct the breathing gas along a water surface to obtain the conditioned breathing gas. The breathing gas humidifier is selectively switchable between the first configuration and the second configuration.

### CROSS REFERENCE TO RELATED APPLICATIONS

This application claims the benefit of priority under 35 U.S.C. § 119 of German Application 10 2022 132 441.9, filed Dec. 7, 2022, the entire contents of which are incorporated herein by reference.

### TECHNICAL FIELD

The present invention pertains to a breathing gas humidifier (respiratory humidifier).

### BACKGROUND

Breathing gas humidifiers are used in ventilators to condition breathing gas intended for ventilating patients to a specific humidity and, if necessary, to a specific temperature.

Breathing gas humidifiers are known in a variety of designs, each optimized for a specific type of therapy.

For example, in the therapy form of invasive ventilation, it is necessary that the flow resistance of the breathing gas humidifier is low. This can be achieved in particular by using an evaporator as a breathing gas humidifier. In an evaporator, water (possibly at a predetermined temperature) delivers moisture to a stream of breathing gas to be humidified, in particular by surface evaporation. However, such and other breathing gas humidifiers used in invasive ventilation are limited in terms of their humidification performance.

For example, high breathing gas flows, e.g. in the range of 40-60 l/min and higher, are used in high-flow therapy. In order to ensure particularly suitable humidification of such a breathing gas flow, breathing gas humidifiers must usually be used in high-flow therapy that are different than the breathing gas humidifiers used in invasive ventilation, as these are limited in terms of their humidification performance. In contrast to invasive therapy, for example, flow resistance plays a subordinate role in this high-flow form of therapy.

In order to provide humidification properties suitable for a particular form of therapy, the humidifier is therefore typically also replaced by another when the form of therapy is changed (cyclically, if necessary). This leads to high handling effort and also increases the number and variety of humidifiers required.

### SUMMARY

It is an object of the invention to provide a breathing gas humidifier whose range of application is increased and whose humidification properties are improved.

These and other objects are attained by breathing gas humidifiers as disclosed herein.

Advantageous embodiments are presented with this disclosure.

In accordance with the invention, there is provided in this respect a breathing gas humidifier comprising a water chamber, the water chamber having a first area for receiving water and having a second area for receiving breathing gas. The breathing gas humidifier further comprises an inlet for supplying breathing gas to the breathing gas humidifier, an outlet for discharging conditioned breathing gas from the breathing gas humidifier, and a bubbler comprising a bubbler element having outlet ports. The breathing gas humidifier is configured to provide, in a first configuration, a fluidic connection between the inlet and the first area via the bubbler element to direct the breathing gas through the water to obtain the conditioned breathing gas. The breathing gas humidifier is further configured, in a second configuration, to provide a fluidic connection between the inlet and the second area to direct the breathing gas along a water surface to obtain the conditioned breathing gas. Further, the breathing gas humidifier is adapted to be selectively switched between the first configuration and the second configuration.

According to the invention, breathing gas means a gas or gas mixture that can be breathed by humans (a breathable gas). The breathing gas may contain oxygen and/or air as components. The breathing gas may also contain other components.

According to the invention, conditioned breathing gas means a breathing gas which has an increased moisture content compared to the supplied breathing gas, for example has a predetermined moisture content, and preferably further has a changed temperature, for example a predetermined temperature.

In this context, a bubbler is understood to be a component that has at least a bubbler element and is set up by means of the bubbler element to distribute the breathing gas through the outlet openings at least in the first area.

According to the invention, a bubbler element is consequently understood as a component comprising the outlet openings, the outlet openings being suitable for distributing the breathing gas at least in the first area, in particular for distributing the breathing gas in water.

Preferably, the outlet openings are arranged (for example by appropriate shape, orientation and/or size of the outlet openings) to distribute the breathing gas uniformly, in the first area in particular in the form of breathing gas bubbles. The outlet openings can be arranged on any sides of the bubbler element, but preferably on the bubbler element underside, i.e. on a side that is at the bottom in the operating state.

The proposed breathing gas humidifier solves the task according to the invention in that the breathing gas humidifier can be switched between two configurations, each of which has different humidification properties.

In the first configuration, a flow path is formed that allows the breathing gas to flow through the water from the inlet via the outlet openings. Through the outlet openings, the breathing gas is introduced into the water, for example in the form of breathing gas bubbles, where it can thus absorb moisture from the surrounding water. Preferably, the temperature of the breathing gas can also be adjusted to the temperature of the water in a rapid manner in the process. In this way, conditioned breathing gas is formed to be discharged from the outlet.

In the second configuration, a flow path is formed that does not allow the breathing gas coming from the inlet to flow through the water, but instead guides the breathing gas through the second area, for example along the surface of the water. In the second configuration, the humidification effect consequently corresponds to a surface evaporator (pass-over evaporator). In this case, the breathing gas absorbs evaporating water in the second area in order to be discharged from the outlet as conditioned breathing gas.

This means that it is not necessary to change the humidifier to switch between therapy forms with different requirements for humidification properties. Instead, it is easy to switch between the two configurations.

Depending on the desired form of therapy, the first configuration can therefore be selected, which has a higher flow resistance but is improved by the bubbler in terms of humidification performance and is advantageous, for example, in high-flow therapy. Alternatively, the second configuration can be selected, which has a reduced flow resistance but is reduced in terms of humidification performance and which is advantageous, for example, in invasive ventilation.

The range of application and the manageability of the humidifier can thus be improved.

Preferably, in addition to the bubbler element, the bubbler further comprises a feed element for guiding the breathing gas to the bubbler element.

The feed element and the bubbler element can be different components from each other that can be assembled to form the bubbler, i.e., the bubbler can be configured as a multi-part unit as a whole. Alternatively, the feed member and the bubbler member may be integrally formed to form a one-piece bubbler.

The feed element, if present, can be configured as a hollow body, i.e., for example, as a tube or a hose.

The water chamber is preferably arranged such that the second area (for receiving breathing gas) is arranged above the first area (for receiving the water) in an operating state.

Preferably, the water chamber is adapted to be placed on a surface, wherein in an operating condition the water arranges itself following gravity in a lower area of the water chamber to form the first area.

In addition to the first area and the second area, the water chamber may further include other areas and/or subdivisions.

Preferably, the water chamber is configured to provide a substantially constant water level. This can be achieved by supplying water, for example by means of a water line (water pipe). For this purpose, the breathing gas humidifier can preferably have a level detector which is in signal connection with a water reservoir, whereby the water reservoir is caused to supply water to the water chamber when the level falls below a setpoint value. Preferably, the level detector is provided as a float, but any other type of level detector is also suitable. Water replenishment without a level detector is also possible, for example, a bottle or bag with a predetermined or predeterminable inflow rate can be connected to the water chamber, for example, via the water line if available.

The optional switchability between the first configuration and the second configuration according to the invention can be achieved in a variety of ways, as will become apparent below.

Preferably, in this respect, the humidifier comprises a position-variable element arranged to establish (and to correspond to) the first configuration in a first position and to establish (and to correspond to) the second configuration in a second position.

In this preferred embodiment, the switchability is consequently achieved by means of the position-variable element that provides a configuration changing means. In this way, the humidifier can be easily configured. Furthermore, since the position-variable element only has to assume two positions, the control of the humidifier is simple.

The position-variable element can be controlled, for example, by an actuator or actuating means comprising a machine element, such as by means of a motor, for example a linear motor, or by means of a pneumatic cylinder, or can be controlled by means of the hand of an operator.

Preferably, the position-variable element is set up to be changed in terms of its position by means of compressed air (pneumatically).

Compressed air is usually already available for ventilators. Thus, little design effort is required in this embodiment to achieve the position variability. For example, the compressed air can be used to operate a machine element such as a pneumatic actuator, in particular a pneumatic lifting cylinder.

In one embodiment, the position-variable element may be a shutoff member that opens toward the second area.

In this respect, a shut-off device is understood to be a component suitable for selectively opening and closing a passage between the bubbler and the second area in terms of fluid flow.

Examples of shut-off devices according to the invention are gate valves, flap valves and other valves, in particular diaphragm valves. Shut-off elements are readily available components and have a variety of designs that can be specifically selected depending on the desired shut-off properties. Therefore, the humidifier can be provided in a particularly flexible and simple manner.

In one embodiment of the position-variable element, the bubbler comprises a first hollow body and a second hollow body, the second hollow body being movable, preferably longitudinally displaceable, relative to the first hollow body to form the position-variable element. Preferably, in this case, the first hollow body and the second hollow body form the feed element, if present.

A hollow body is understood to be a body suitable for carrying breathing gas and at least partially enclosing a cavity, without being limited in terms of its material, shape (including cross-sectional shape) or other properties. In the simplest case, the hollow body has a cylindrical shape.

For example, the first hollow body and the second hollow body may be concentrically interlocked. The first hollow body and/or the second hollow body may be provided, for example, as tubes and/or as hoses.

The first and/or the second hollow body preferably have an opening towards the second area. The opening is preferably closed in the first position by at least partial overlapping of the hollow bodies. The opening is preferably open in the second position.

In this embodiment of the position-variable element (or variable-position element), which is an alternative to the shut-off element, the opening to the second area is thus opened by moving, preferably by displacing, the second hollow body relative to the first hollow body.

In the second position, the opening is thus permeable to the breathing gas towards the second area. In the first position, the opening is closed and consequently not permeable to the breathing gas, so that the breathing gas flows in the direction of the bubbler element in this position.

Thus, the humidifier can be provided in a simple configuration.

In a preferred and alternative embodiment for providing a position-variable element, the bubbler comprises a first fluid path and separately a second fluid path, wherein the first fluid path corresponds to the first configuration and the second fluid path corresponds to the second configuration.

In this embodiment, the ability to switch between the first and second configurations according to the invention is achieved by providing two separate (first and second) fluid paths through which the breathing gas can flow selectively by suitable switching means (a position-variable element). This can advantageously reduce the number of moving parts, which simplifies the design of the breathing gas humidifier.

A valve, for example, is suitable as a switching means. The valve can be actuated by an actuating means comprising a machine element or manually. Further, the switching means can be provided by having the inlet have two different ports (for example, a first inlet and a second inlet), each connected to one of the two fluid paths. By connecting a breathing gas tube to one port (for example, the first inlet), the first fluid path can be fluidically connected, and by connecting to the other port (for example, the second inlet), the other fluid path can be fluidically connected.

The switching means can also be configured in a different way than described above.

In another preferred embodiment, the bubbler element may be adapted to form the position-varying element.

Thus, the position-variable element can be provided in a particularly simple manner by having the bubbler element form the position-variable element.

All that is required for this is that the first position of the bubbler element corresponds to the first configuration and the second position of the bubbler element corresponds to the second configuration.

This can be achieved by arranging the bubbler element in the first position in the first area to allow the breathing gas to flow through the water and by changing its position in the second position in the second area to prevent the breathing gas to flow through the water. Preferably, the breathing gas thereby flows through the outlet openings of the bubbler element in both the first and second configurations.

This embodiment also offers the advantage that the outlet openings can also participate in the breathing gas distribution in the water chamber in the second configuration, since the breathing gas can preferably flow through the outlet openings in both configurations, as described. Consequently, the humidification characteristics can thus also be influenced in the second configuration, since, for example, depending on the desired flow characteristics, the outlet openings can be arranged directionally to favor laminar flow in the second area or, for example, can be arranged non-directionally to favor turbulent flow.

The bubbler element can be configured to be longitudinally displaceable or pivotable relative to the water chamber, for example, in order to achieve position variability.

For example, the bubbler element can be arranged on a height-variable element, for example on a height-variable feed element, so that the bubbler element and height-variable element form a structural unit. For this purpose, for example, the feed element can be accommodated in the water chamber so as to be displaceable relative thereto, for example by accommodating the feed element by means of a plain bearing on or in the water chamber.

Preferably, in one embodiment, the bubbler element has a plurality of hollow bodies branching off in a star shape.

This allows the bubbler element to contribute to a particularly even distribution of breathing gas in the water chamber.

By hollow bodies branching off in a star shape it is understood that hollow bodies extend in a radial direction with respect to a longitudinal axis of the bubbler, in particular of the feed element, preferably evenly spaced. The tips or the distal ends of the branching hollow bodies thus correspond to the prongs of a star in the geometric sense. At least the hollow bodies have the outlet openings.

In this respect, hollow cylinders are particularly suitable as hollow bodies, but essentially any configuration is possible.

In a further embodiment, the bubbler element is preferably formed as a perforated plate.

According to the invention, a perforated plate (an apertured plate) is understood to be both a planar body, i.e. a body with a substantially constant thickness extension and consequently with a substantially constant cross-section, and a body with a non-constant thickness extension and consequently with a non-constant cross-section. In this case, at least the body of the perforated plate has outlet openings.

By providing the bubbler element as a perforated plate, the distribution of breathing gas in the water chamber can easily take place over a large area, for example over the entire cross-section of the water chamber, and the humidification performance can be improved at least in the first configuration.

Preferably, the perforated plate is essentially flat, funnel-shaped and/or curved, especially wave-shaped (with an undulating form).

Combinations of the above forms are also possible.

Funnel-shaped refers to a shape of the perforated plate such that it at least partially has the shape of at least one cone and/or at least one truncated cone. The cone and/or the truncated cone has at least part of the outlet openings at its apex and/or at its frustum and/or on the lateral surface.

Wave-shaped refers to a shape of the perforated plate such that the cross-section of the perforated plate corresponds to a wave in at least one direction. Particularly preferably, the perforated plate is rotationally symmetrical.

Thus, the properties of the perforated plate can be optimized in relation to the requirements of the humidifier. For example, if a flat perforated plate is provided, the manufacturing costs can be reduced due to the simple geometry. On the other hand, if a funnel-shaped or curved perforated plate is provided, the surface area available for the delivery of breathing gas can be increased and the humidification performance can be further improved.

Providing a shape of the perforated plate that deviates from a flat shape, such as a funnel shape or wave shape, can advantageously prevent air from accumulating under the perforated plate if it is arranged horizontally in the water chamber.

Preferably, the bubbler element has a heating element disposed therein or thereon.

In this way, the temperature of the conditioned breathing gas can be specifically influenced.

In one variant, the heating energy required to operate the heating element can be supplied by a separate energy source. In another variant, a heater of an expiratory branch of a ventilator can be used to operate the heating element, especially in an operating state of the ventilator in which only inspiratory heating is required. This is the case, for example, in high-flow therapy, which is a specific use case for the second configuration.

Preferably, the water chamber is configured to be heated by means of a heating source, in particular by means of a heating plate. For example, the water chamber comprises a thermally conductive side and/or base plate for this purpose.

In this way, the evaporation behavior of the water can be specifically influenced. Furthermore, the breathing gas humidifier is in this way compatible with ventilation systems which already have a heating plate. However, compared to known respiratory systems that operate in passover mode and are set up to be heated by means of a heating plate, the present invention offers the advantage that, due to the provision of a bubbler, there is a high efficiency of heat transfer from the heated water into the breathing gas, which is not conventionally the case.

Consequently, the heating power and correspondingly the water temperature in the breathing gas humidifier or water chamber of the present invention can be advantageously reduced compared to known systems in order to achieve a comparable humidity and, if necessary, temperature in the conditioned breathing gas. Consequently, the risk of hot-shots, burns or scalds can be reliably reduced.

In this embodiment, it is also preferred that the bubbler element or its exit ports in the second configuration is (are) positioned close enough to a heat transfer surface of the water chamber to allow significant heat transfer from the heat source into the breathing gas.

Particularly preferably, the bubbler element has a heating element, and the water chamber is also configured to be heated by means of a heat source. This combination allows the temperature and humidity of the conditioned breathing gas to be influenced in a particularly targeted manner.

These and other features and advantages of the present invention are also apparent from the following description of figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1a is a perspective partially transparent view showing an embodiment of the humidifier in the second configuration;

FIG. 1b is a perspective partially transparent view showing an embodiment example of the humidifier in the first configuration;

FIG. 2a is a cross-sectional view showing an embodiment of a shut-off device in a first position and also schematically showing an actuating means;

FIG. 2b is a cross-sectional view showing an embodiment example of a shutoff member in a second position and also schematically showing an actuating means;

FIG. 3a is a cross-sectional view showing an embodiment of a shut-off device in a first position;

FIG. 3b is a cross-sectional view showing an embodiment of a shutoff member in a second position;

FIG. 4 is a perspective view showing an embodiment of a bubbler element;

FIG. 5 is a perspective view showing an embodiment of a bubbler element;

FIG. 6 is a perspective view showing an embodiment of a bubbler element;

FIG. 7 is a perspective view showing an embodiment of a bubbler element;

FIG. 8 is a perspective view showing an embodiment of a bubbler element;

FIG. 9 is a perspective partially transparent view showing an embodiment of the breathing gas humidifier with a first fluid path and a second fluid path and also schematically showing a switching means;

FIG. 10 is a perspective partially transparent view showing example of the humidifier with a water line (water pipe).

### DESCRIPTION OF PREFERRED EMBODIMENTS

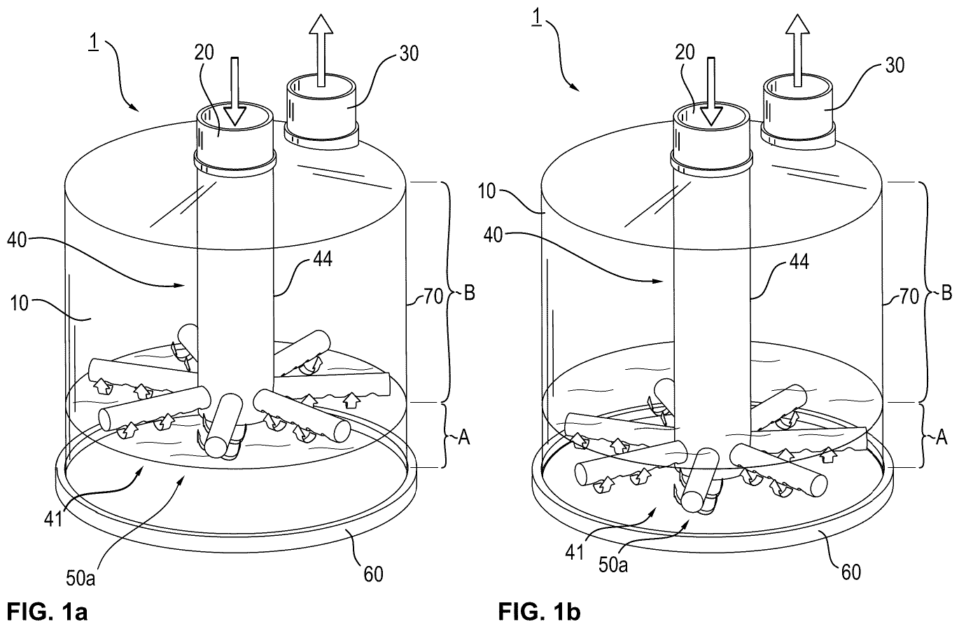

Referring to the drawings, as shown in FIGS. 1a, 1b, 9 and 10, a breathing gas humidifier 1 is provided according to the invention.

The breathing gas humidifier 1 has a water chamber 10, as can be seen in FIGS. 1a, 1b, 9 and 10.

The water chamber 10 includes, for example, a substantially cylindrical or conical base body bounded by at least one chamber wall 70. As shown, the water chamber 10 may include a base plate 60 that may be offset from the chamber wall 70, for example, in that the base plate 60 may protrude in a radial direction as shown. The chamber wall 70 may be made of any material, such as a plastic or glass.

The water chamber 10 has a first area A for holding water and a second area B for holding breathing gas. Further subdivisions of the water chamber 10 are possible.

In an operating state, the water chamber 10 is preferably arranged, as shown in FIGS. 1a, 1b, 9 and 10, in such a way that the (water-receiving) first area A is arranged below the (breathing gas-receiving) second area B. In the operating state, the first area A of the water chamber 10 is, for example, filled with a preferably predetermined amount of water.

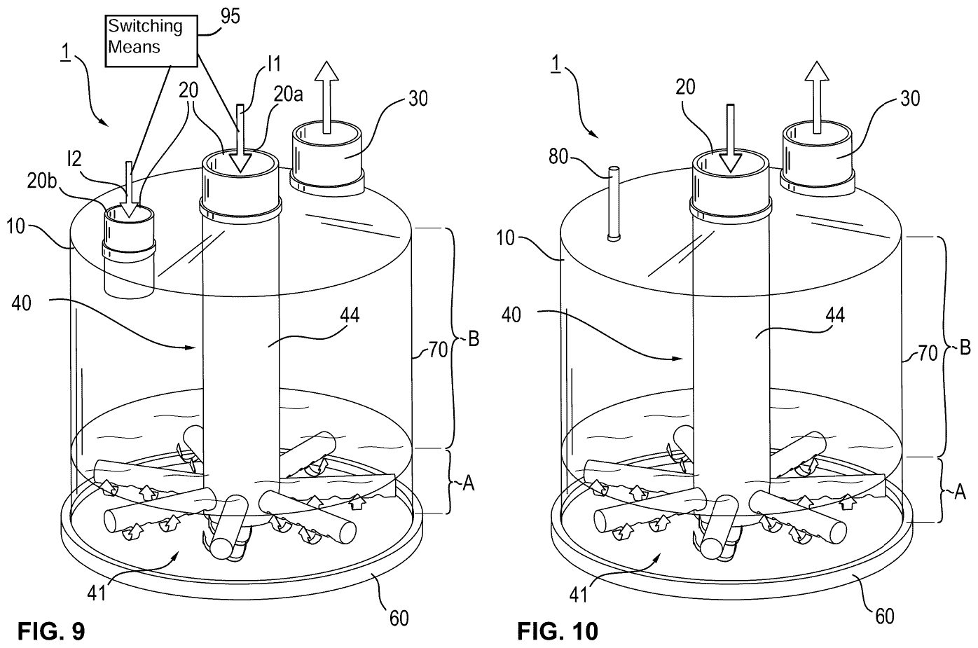

The breathing gas humidifier 1 further comprises an inlet 20 for supplying breathing gas to the breathing gas humidifier 1 and an outlet 30 for discharging conditioned breathing gas from the breathing gas humidifier 1. The breathing gas humidifier may have a plurality of inlets 20, as can be seen in FIG. 9 in the form of a first inlet 20a and a second inlet 20b. What has been said for the inlet 20 applies analogously in the case of multiple inlets 20a, 20b. The breathing gas humidifier 1 can likewise have several outlets 30.

The inlet 20 and the outlet 30 can be implemented in a variety of ways, in particular depending on the components to be connected, for example a ventilator. For example, a breathing gas tube (hose) of a ventilator can be connected to the inlet 20 in order to supply breathing gas. The inspiratory line of a breathing mask, a nasal probe, a tube or the like can be connected to the outlet 30, which provides the conditioned breathing gas, for example.

The inlets 20 and outlets 30 shown in FIGS. 1a, 1b, 9 and 10 are schematically configured as cylindrical passages through the chamber wall 70.

As shown in FIGS. 1a, 1b, 9 and 10, the breathing gas humidifier 1 includes a bubbler 40 having a bubbler element 41 with outlet ports. In a first configuration, the bubbler 40 is arranged to provide a fluidic connection between the inlet 20 and the first area A via the bubbler element 41 to pass the breathing gas through the water to obtain the conditioned breathing gas.

Thus, in the first configuration, the breathing gas is passed through the water in the first area A, where it emerges from the outlet openings. The exiting breathing gas is indicated with arrows in FIGS. 1a, 1b and 4 to 10. The breathing gas is introduced into the water and humidified, for example, according to the number, size and shape of the outlet openings of the bubbler element 41. The breathing gas bubbles that are preferably formed rise in the direction of the second area B in the example shown, thus forming the conditioned breathing gas which can be discharged via the outlet 30.

The bubbler 40 is also configured to provide a fluidic connection between the inlet 20 and the second area B in a second configuration so as to direct the breathing gas along a water surface to obtain the conditioned breathing gas.

Thus, in the second configuration, the breathing gas is not passed through the water in the first area A, but only through the second area B. The breathing gas is accordingly directed along the water surface, where it accumulates water vapor and is discharged as conditioned breathing gas via the outlet 30.

According to the invention, the breathing gas humidifier 1 is further adapted to be selectively switched between the first configuration and the second configuration. The features providing the first configuration, the second configuration and the selective switching comprise a configuration changing means.

As shown in all figures, in addition to the bubbler element 41, the bubbler 40 may also include a delivery element 44 that is used to deliver the breathing gas to the bubbler element 41. The supply member 44 may be, for example, a tube or tube.

The outlet openings of the bubbler element 41 can be arranged on any sides of the bubbler element 41, but preferably on its underside, i.e. on a side that is at the bottom in the operating state.

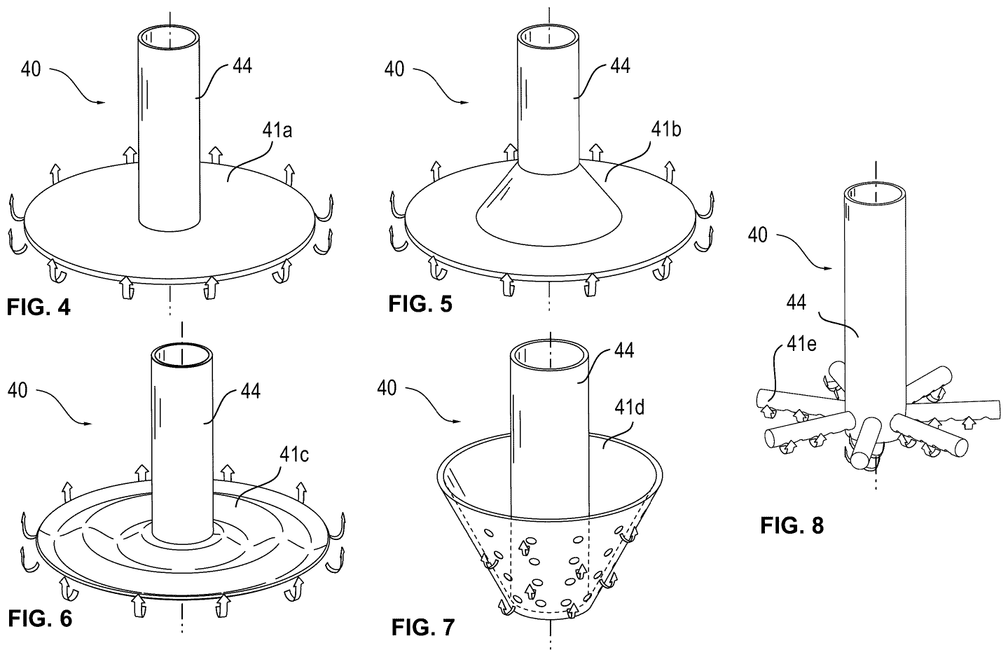

The bubbler element 41 can be implemented in a variety of ways. In this respect, FIGS. 4 to 8 show exemplary embodiments of bubbler elements 41a to 41e.

In an example shown in FIG. 8, the bubbler element 41 may comprise a plurality of star-shaped branching hollow bodies 41e, as described at the outset. In FIGS. 1a, 1b, 9, and 10, the bubbler element 41 is shown in this respect as an example with hollow bodies 41e branching off in a star shape, although any other shape of bubbler element 41 is possible for each of the embodiments.

In FIGS. 4 to 7, embodiments of the bubbler element 41 are shown in which the bubbler element 41 is formed as a perforated plate 41a, 41b, 41c, 41d. The perforated plate 41a, 41b, 41c, 41d may have a variety of shapes, as described at the outset.

In the examples of FIGS. 4 to 7, it is shown that the perforated plate 41a, 41b, 41c, 41d can extend essentially flat (FIG. 4) and/or be funnel-shaped (FIGS. 5, 7) and/or curved, in particular wave-shaped (FIG. 6). In the embodiments of FIGS. 4-7, the number of apertures of the perforated plates may be selected. The embodiments of FIGS. 4-6 may be provided as a barrier plate 41a, 41b, 41c, with a peripheral edge spaced from the chamber wall 70 and no apertures, such that breathing gas flows around the peripheral edge. Apertures of the perforated plate 41a, 41b, 41c, may be combined with the peripheral edge spaced from the chamber wall 70 or the peripheral edge may extend to the chamber wall 70, such that breathing gas flows only through apertures of the perforated plate 41a, 41b, 41c.

In the example of a perforated plate 41b shown in FIG. 5, a combination of a funnel shape and a flat shape is present. Here, an outer portion of the perforated plate 41b is flat and an inner portion is funnel-shaped, as shown. In the example shown, a truncated cone extends upwardly tapering from the planar base area.

In the example shown in FIG. 6, the perforated plate 41c is wave-shaped, and is a substantially rotationally symmetrical perforated plate 41c, wherein a cross-section in a plane of the central axis of the perforated plate 41c corresponds to the shape of a wave.

In the example shown in FIG. 7, the perforated plate 41d is funnel-shaped, with the perforated plate 41d extending downwardly in the form of a truncated cone.

In some embodiments, as shown in FIGS. 1a, 1b, 2a, 2b, 3a and 3b, switchability between the first configuration and the second configuration according to the invention is achieved by the breathing gas humidifier 1 comprising a position-variable element 50a, 50b, 50c arranged to correspond in a first position to the first configuration and in a second position to the second configuration. The configuration changing means in this case comprises the position-variable element.

In an embodiment shown in FIGS. 1a and 1b, the bubbler element 41 is adapted to form the position-variable element 50a of the configuration changing means.

For this purpose, the bubbler 40 as a whole or only the bubbler element 41 may be longitudinally displaceable (i.e., displaceable along a longitudinal axis of the bubbler 40 or a longitudinal axis of the water chamber 10, respectively), for example. In this regard, the bubbler element 41 provides (establishes) the first configuration in the first position shown in FIG. 1b, in that the bubbler element 41 is located in the first area A in the first position. By changing the position, for example by longitudinal displacement, the bubbler element 41 can be arranged in the second position in the second area B to provide (establish) the second configuration. By changing the position of the bubbler element 41, it is thus possible to switch between the two configurations.

The position variability of the bubbler element 41, preferably the longitudinal displaceability, can be achieved in a variety of ways. One example is the provision of the bubbler element 41 at a height-variable feed element 44, as shown in FIGS. 1a, 1b and described in detail above. In this case, the bubbler 40 is arranged as a unit longitudinally displaceable relative to the water chamber 10, for example by slidingly receiving the bubbler 40 in the water chamber 10.

In the case of a position-variable element 50a, 50b, 50c in general, and in particular in the case of a bubbler element 41 as a position-variable element 50a, it is preferred that the position change can be achieved by an actuating means 90 of the configuration changing means, such as a machine element, such as by a motor. However, it is also possible to change the position manually by means of an operator's hand (a manually operatable actuating means). In all embodiments, the position-variable element 50a, 50b, 50c can, for example, be set up to be changed with respect to its position by means of compressed air (the actuating means 90 is pneumatically driven).

FIGS. 3a, 3b show a further embodiment of a position-variable element, which in this respect represents an alternative to the embodiment according to FIGS. 1a, 1b. In this case, the position-variable element can be a shut-off element 50c that can be opened toward the second area B.

In an exemplary embodiment shown in FIGS. 3a, 3b, a flap can be provided as a shut-off element 50c, which can be opened towards the second area B via a joint and thus opens or closes an opening O in the feed element 44.

When the shut-off element 50c is in the first position, i.e., closed in this example, the breathing gas flows towards the bubbler element 41, as indicated by the arrows in FIG. 3a. Thus, the breathing gas humidifier 1 is in the first configuration.

When the shut-off element 50c is in the second position, i.e., open in this example, the breathing gas flows through the opening O towards the second area B, as indicated by the arrows in FIG. 3b. Thus, the breathing gas humidifier is in the second configuration.

As indicated by dash lines in FIGS. 3a, 3b, the shut-off element 50c may be arranged just above the first area A. By opening the shut-off element 50c, the breathing gas consequently flows through the opening O along the water surface as shown in FIG. 3b. Since breathing gas thus no longer escapes from the bubbler element 41, in this state the water in the feed element 44 can rise to the height corresponding to the dash lines.

Thus, by opening or closing the shut-off element 50c, it is possible to selectively switch between the first and second configurations.

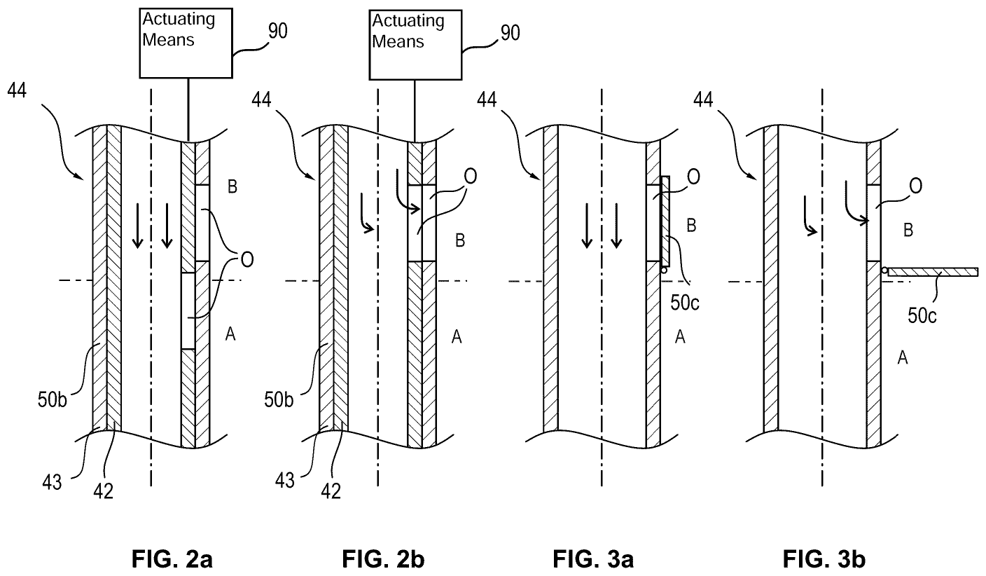

FIGS. 2a, 2b show a further embodiment of a position-variable element, which in this respect represents an alternative to the two embodiments shown in FIGS. 1a, 1b and FIGS. 2a, 2b.

In this case, the feed element 44 comprises a first hollow body 42 and a second hollow body 43, wherein in the example shown the second hollow body 43 is movable, in this case displaceable, relative to the first hollow body 42 and thus forms the position-variable element 50b. The hollow bodies 42, 43 may be tubes, for example, which are arranged concentrically in the example shown.

The first hollow body 42 and/or the second hollow body 43 may have an opening O toward the second area B. In the example shown, both hollow bodies 42, 43 have an opening O.

As shown in FIG. 2a, the opening O may be closed in the first position by overlapping the hollow bodies 42, 43. In the first position shown in FIG. 2a, as indicated by the arrows, the breathing gas flows through the feed element 44 towards the bubbler element 41 and thus into the first area A to provide the first configuration,

In the second position shown in FIG. 2b, the opening O can be opened by moving one or both hollow bodies 42, 43 relative to each other via an actuating means (actuator) 90. In this position, as indicated by the arrows, the breathing gas flows through the opening(s) O toward the second area B to provide the second configuration.

As indicated by dash lines in FIGS. 2a, 2b, the opening(s) O may be located just above the first area A when open. By opening the opening(s) O, the breathing gas flows through the opening(s) O along the water surface as shown in FIG. 2b. Since breathing gas thus no longer escapes from the bubbler element 41, in this state the water in the feed element 44 can rise to the height corresponding to the dash lines.

Thus, by moving one or both hollow bodies 42, 43 relative to each other, it is possible to selectively switch between the first and second configurations.

In an embodiment shown in FIG. 9 and alternative to the embodiments shown in FIGS. 1a, 1b, 2a, 2b, 3a, 3b, the ability to switch between the first configuration and the second configuration according to the invention is achieved by the bubbler 40 comprising a first fluid path I1 and, separately, a second fluid path I2.

In the embodiment shown in FIG. 9, this is realized by the inlet 20 having two different inlets 20a, 20b. A first inlet 20a is connected to the first fluid path I1 and a second inlet 20b is connected to the second fluid path I2. The first fluid path I1 corresponds to (and establishes/provides) the first configuration and the second fluid path I2 corresponds to (and establishes/provides) the second configuration.

This is achieved, for example, by the first fluid path I1 being arranged to fluidically connect the first inlet 20a to the bubbler element 41 to direct the breathing gas into the first area A.

Further, for example, the second fluid path I2 may be arranged to fluidically connect the second inlet 20b indirectly or directly to the second area B to direct breathing gas into the second area B.

For example, by connecting a breathing gas tube to the first inlet 20a, fluid can flow through the first fluid path I1, and by connecting it to the second inlet 20b, fluid can flow through the second fluid path I2.

Consequently, the breathing gas optionally flows through the first fluid path I1 into the first area A or through the second fluid path I2 into the second area B.

Switching between the first configuration and the second configuration can be accomplished by a switching means 95, for example, by manually reconnecting the breathing gas tube (a manual switching means 95) or by using an appropriate component as the switching means 95 to control the flow of the breathing gas, such as a valve.

Not shown in the figures is that in all embodiments, the bubbler element 41 may have a heating element disposed therein or thereon.

In the embodiments shown, the water chamber 10 may be arranged to be heated by means of a heating source, in particular by means of a heating plate. For example, the base plate 60 is thermally conductive for this purpose and can be arranged on a heating plate.

In an embodiment shown in FIG. 10, the breathing gas humidifier 1 may additionally comprise a water line 80 through which water may be supplied to the water chamber 10. Such a water line 80 may be provided in any embodiment.

The water line 80, if present as shown in FIG. 10, may be, for example, a cylindrical passageway through the chamber wall 70 into the water chamber 70.

In embodiments of the humidifier as exemplified in FIGS. 1a, 1b, 2a, 2b, 3a, 3b, 9, and 10 and described herein, the bubbler 40 may be as shown in FIG. 4, 5, 6, 7, or 8 and described herein.

All of the features disclosed herein may be combined, provided that this is not contradictory or that this relates to alternatives.

While specific embodiments of the invention have been shown and described in detail to illustrate the application of the principles of the invention, it will be understood that the invention may be embodied otherwise without departing from such principles.

### LIST OF REFERENCE CHARACTERS

1 Breathing gas humidifier
10 Water chamber
20 Inlet
20a First inlet
20b Second inlet
30 Outlet
40 Bubbler
41 Bubbler element
41a, 41b, 41c, 41d Perforated plate
41e Star-shaped branching hollow body
42 First hollow body
43 Second hollow body
44 Feed element
50a, 50b, 50c Position-variable element
50b, 50c Shut-off device
60 Floor plate
70 Chamber wall
80 Water line
90 Actuating means
95 Switching means
A First area
B Second area
I1 First fluid path
I2 Second fluid path
O Opening

What is claimed is:

1. A breathing gas humidifier comprising:
a water chamber comprising a first area for receiving water and a second area for receiving breathing gas;
an inlet for supplying breathing gas to the humidifier;
an outlet for discharging conditioned breathing gas from the humidifier; and
a bubbler comprising a bubbler element with outlet openings,
wherein the breathing gas humidifier is configured to provide a first configuration with a fluidic connection between the inlet and the first area via the bubbler element to pass the supplied breathing gas through water in the first area so as to obtain the conditioned breathing gas,
wherein the breathing gas humidifier is configured to provide a second configuration with a fluidic connection between the inlet and the second area to direct the breathing gas along a water surface of water in the first area so as to obtain the conditioned breathing gas, and
wherein the breathing gas humidifier is configured to be selectively switched between the first configuration and the second configuration.

2. A breathing gas humidifier according to claim 1, further comprising a position-variable element configured to provide the first configuration in a first position and to provide the second configuration in a second position.

3. A breathing gas humidifier according to claim 2, wherein the position-variable element is arranged to be changed with respect to a position of the position-variable element by means of compressed air.

4. A breathing gas humidifier according to claim 2, wherein the position-changing element comprises a shut-off element openable towards the second area.

5. A breathing gas humidifier according to claim 2, wherein the bubbler comprises:
a first hollow body; and
a second hollow body, the second hollow body being movable relative to the first hollow body to form the position-variable member,
wherein the first hollow body and/or the second hollow body has an opening towards the second area,
wherein the opening is closed in the first position by an overlapping of the hollow bodies, and
wherein the opening is open in the second position.

6. A breathing gas humidifier according to claim 1,
wherein the bubbler comprises a first fluid path and a second fluid path, which second fluid path is separate from the first fluid path,
wherein the first fluid path establishes the first configuration, and the second fluid path establishes the second configuration.

7. A breathing gas humidifier according to claim 2, wherein the bubbler element is adapted to form the position-variable element.

8. A breathing gas humidifier according to claim 7, wherein the bubbler element comprises a plurality of hollow bodies branching off in a star shape.

9. A breathing gas humidifier according to claim 7, wherein the bubbler element is formed as a perforated plate.

10. A breathing gas humidifier according to claim 9, wherein the perforated plate extends essentially flat, has a funnel shape, a curved shape and/or a wave shape.

11. A breathing gas humidifier comprising:
a water chamber comprising a first area for receiving water and a second area for receiving breathing gas;
an inlet for supplying breathing gas to the humidifier;
an outlet for discharging conditioned breathing gas from the humidifier;
a bubbler comprising a bubbler element with outlet openings; and
configuration changing means for providing a first configuration with a fluidic connection between the inlet and the first area via the bubbler element to pass the supplied breathing gas through water in the first area so as to obtain the conditioned breathing gas, and for providing a second configuration with a fluidic connection between the inlet and the second area to direct the breathing gas along a water surface of water in the first area so as to obtain the conditioned breathing gas, and for being selectively switched between the first configuration and the second configuration.

12. A breathing gas humidifier according to claim 11, wherein the configuration changing means comprises a position-variable element configured to provide the first configuration in a first position and to provide the second configuration in a second position.

13. A breathing gas humidifier according to claim 12, wherein the configuration changing means comprises an actuating means to change the position of the position-variable element between the first position and the second position by means of compressed air.

14. A breathing gas humidifier according to claim 12, wherein the position-changing element comprises a shut-off element to change the flow state between an open state and a closed state towards the second area.

15. A breathing gas humidifier according to claim 12, wherein the bubbler comprises:
a first hollow body; and
a second hollow body, the second hollow body being movable relative to the first hollow body to form the position-variable member,
wherein the first hollow body and/or the second hollow body has an opening towards the second area,
wherein the opening is closed in the first position by an overlapping of the hollow bodies, and
wherein the opening is open in the second position.

16. A breathing gas humidifier according to claim 11,
wherein the bubbler comprises a first fluid path and a second fluid path, which second fluid path is separate from the first fluid path,
wherein the first fluid path establishes the first configuration, and the second fluid path establishes the second configuration and fluid flow is selectively switched between the first fluid path and the second fluid path.

17. A breathing gas humidifier according to claim 12, wherein the bubbler element includes the position-variable element.

18. A breathing gas humidifier according to claim 17, wherein the bubbler element comprises a plurality of hollow bodies branching off in a star shape.

19. A breathing gas humidifier according to claim 17, wherein the bubbler element is formed as a perforated plate and/or a barrier plate with a peripheral flow edge.

20. A breathing gas humidifier according to claim 19, wherein the perforated plate extends essentially flat, has a funnel shape, a curved shape and/or a wave shape.
